(19) United States
(12) Patent Application Publication
(10) Pub. No.: US 2011/0124716 A1
(43) Pub. Date: May 26, 2011

(54) ULTRASOUND-ASSISTED GENE TRANSFER TO SALIVARY GLANDS

(75) Inventor: Michael J. Passineau, Franklin Park, PA (US)

(73) Assignee: ALLEGHENY-SINGER RESEARCH INSTITUTE, Pittsburgh, PA (US)

(21) Appl. No.: 12/860,559

(22) Filed: Aug. 20, 2010

Related U.S. Application Data
(60) Provisional application No. 61/235,379, filed on Aug. 20, 2009.
(60) Provisional application No. 61/373,458, filed on Aug. 13, 2010.

(51) Int. Cl. A61K 48/00; A61P 9/12

(52) U.S. Cl. 514/44 R

(57) ABSTRACT

Methods and compositions for delivery of genetic material to salivary glands using ultrasound and microbubbles are described herein.

### CROSS-REFERENCE TO RELATED APPLICATIONS

This application claims priority to U.S. Provisional Application No. 61/235,379, filed Aug. 20, 2009 entitled “Ultrasound-Assisted Gene Transfer to Salivary Glands” and U.S. Provisional Application No. 61/373,458, filed Aug. 13, 2010 entitled “Ultrasound-Assisted Gene Transfer to Salivary Glands,” which are incorporated herein by reference in their entirety.

### GOVERNMENT INTERESTS

This invention was made with Government support under grant No.: 3R00DE018188-0351 awarded by the NIH. The Government has certain rights in the invention.

### PARTIES TO A JOINT RESEARCH AGREEMENT

Not applicable

### INCORPORATION BY REFERENCE OF MATERIAL SUBMITTED ON A COMPACT DISC

Not applicable

### BACKGROUND

Not applicable

### SUMMARY

Various embodiments of the invention are directed to methods for delivering genetic material to salivary glands including, for example, the steps of preparing a solution of about 5% to about 30% microbubbles and about 10 μg to about 100 μg genetic material or about 0.1 μg/μl to about 5 μg/μl genetic material in the final genetic material/microbubble solution; contacting one or more salivary glands with the solution; and applying ultrasound to the salivary glands. In particular embodiments, salivary glands may be any of the parotid salivary gland, the submandibular salivary gland, the sublingual salivary gland or combinations thereof. In some embodiments, the genetic material may be a viral vector, and in other embodiments, the genetic material may be a non-viral vector. In certain embodiments, the genetic material may include a gene from which a therapeutically effective amount of an exogenously expressed protein can be produced.

In some embodiments, the method may further include the step of preparing a solution which includes combining the genetic material with microbubbles in a physiologically acceptable solution. In particular embodiments, the physiologically acceptable solution may include a liquid such as, for example, saline, phosphate buffered saline, aqueous glucose, human serum albumin, and combinations thereof, and in certain embodiments, the physiologically acceptable solution may be phosphate buffered saline.

In some embodiments, the microbubbles may include a shell of a material such as, for example, albumin, lipids, phospholipids, polymers, and combinations thereof, and in other embodiments, the microbubbles may include a gas such as, for example, air, oxygen, nitrogen, noble gases, sulfur hexafluoride, perfluoride, and combinations thereof. In such embodiments, the microbubbles may have a diameter of from about 1 μm to about 5 μm.

In some embodiments, the step of contacting may include injecting the solution into one or more salivary glands, and in other embodiments, the step of contacting may include infusing the material to the gland via the salivary duct.

In certain embodiments, the step of applying ultrasound may include applying diagnostic ultrasound, therapeutic ultrasound, focused ultrasound, high intensity focused ultrasound, or combinations thereof to the one or more salivary glands. In some embodiments, the ultrasound may include ultrasound energy having an acoustic power density of from about 0.05 W/cm² to about 10 W/cm², and in other embodiments, the ultrasound may include ultrasound energy having a frequency of from about 0.015 MHz to about 10 MHz. In still other embodiments, applying the ultrasound may occur for a time period of from about 30 seconds to about 3 minutes. In particular embodiments, greater than 20% of the genetic material may be delivered to the cytosol of cells of the one or more salivary glands. In certain embodiments, the method may be repeated, and further embodiments may include the step of administering a second form of therapy.

Other embodiments of the invention include a method for treating a disease that may include the steps of preparing a solution of about 15% microbubbles and about 10 μg to about 100 μg of the non-viral vector; contacting one or more salivary glands with the solution; and applying ultrasound to the salivary glands. In some embodiments, the steps of contacting one or more salivary glands with the solution and applying ultrasound to the salivary glands may be repeated at regular intervals for a period of time. In various embodiments, the expression levels of a protein expressed from the non-viral vector may be maintained over the period of time as a result of repeating the steps of contacting one or more salivary glands with the solution and applying ultrasound to the salivary glands. In certain embodiments, the non-viral vector may include a therapeutic protein sequence.

Yet other embodiments of the invention include a method for treating a disease including: preparing a solution of about 5% to about 30% microbubbles and about 10 μg to about 100 μg of the non-viral vector or about 10% to about 25% genetic material; contacting one or more salivary glands with the solution; applying ultrasound to the salivary glands; and repeating the steps of contacting one or more salivary glands with the solution and applying ultrasound to the salivary glands at regular intervals over an extended period of time. In some embodiments, the expression levels of a protein expressed from the non-viral vector may be maintained over the period of time as a result of repeating the steps of contacting one or more salivary glands with the solution and applying ultrasound to the salivary glands. In some embodiments, the extended period of time may be greater than 1 year, and in other embodiments, the extended period of time may be greater than 5 years. In still other embodiments, the extended period of time is the lifetime of the subject.

Further embodiments are directed to a pharmaceutical composition including about 5% to about 30% microbubbles and about 10% to about 25% genetic material. In some embodiments, the pharmaceutical composition may further include a physiologically acceptable solution such as, but not limited to, a liquid selected from saline, phosphate buffered saline, aqueous glucose, human serum albumin, and combinations thereof. In particular embodiments, the physiologically acceptable solution may be phosphate buffered saline. In certain embodiments, the solution may include about 10% to about 20% microbubbles, and in other embodiments, the solution may include about 15% microbubbles. In some embodiments, the microbubbles may include a shell of a material such as, for example, albumin, lipids, phospholipids, polymers, and combinations thereof, and the micobubbles may include a gas such as atmospheric air, oxygen, nitrogen, noble gases, sulfur hexafluoride, perfluoride, and combinations thereof. In other embodiments, the microbubbles may have a diameter of from about 1 μm to about 5 μm.

### DESCRIPTION OF THE DRAWINGS

The file of this patent contains at least one photograph or drawing executed in color. Copies of this patent with color drawing(s) or photograph(s) will be provided by the Patent and Trademark Office upon request and payment of necessary fee.

For a fuller understanding of the nature and advantages of the present invention, reference should be made to the following detailed description taken in connection with the accompanying drawings, in which:

FIG. 1 is a line graph showing Luciferase expression in mouse salivary glands over time following ultrasound mediated administration of solution containing 10 vol. %, 15 vol. %, 20 vol. %, 30 vol. %, and 50 vol. % microbubbles and 50 μL of a non-viral vector including the firefly Luciferase gene.

FIG. 2 is a photograph showing exogenous expression of Luciferase in the salivary glands of a mouse.

FIG. 3 is a photograph showing exogenous expression of Luciferase in the salivary glands of a mouse.

FIG. 4 demonstrates oral cannulation of the submandibular duct.

FIG. 5 shows UAGT with Luciferase in Salivary Glands. (A) Total flux at various timepoints relative to Adenoviral gene delivery. Background was observed to average ˜30,000 photons/sec. (B) Pseudocolored image of photon intensity overlayed on a photograph at 14 days post-UAGT.

FIG. 6 shows an exemplary high frequency echocardiography of PAH (A) and normal (B) animals. RV=Right Ventricle; LV=Left Ventricle.

FIG. 7 depicts an exemplary 3-D Micro-CT reconstruction of the vascular tree of one lobe of a rat lung showing macro(yellow) and micro(pink).

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims.

It must be noted that, as used herein, and in the appended claims, the singular forms “a,” “an,” and “the” include plural reference unless the context clearly dictates otherwise. Unless defined otherwise, all technical and scientific terms used herein have the same meanings as commonly understood by one of ordinary skill in the art. Although any methods similar or equivalent to those described herein can be used in the practice or testing of embodiments of the present invention, the preferred methods are now described. All publications and references mentioned herein are incorporated by reference. Nothing herein is to be construed as an admission that the invention is not entitled to antedate such disclosure by virtue of prior invention.

As used herein, the term “about” means plus or minus 10% of the numerical value of the number with which it is being used. Therefore, about 50% means in the range of 45%-55%.

Embodiments of the invention are generally directed to methods for delivering genetic material to salivary glands using microbubbles and ultrasound sonoporation, and various materials and compositions such as microbubbles and genetic material that may be delivered to salivary glands by sonoporation. In various embodiments, the method may include the steps of preparing a solution of microbubbles and genetic material, contacting one or more target organs or glands of a subject with the mixture, and applying ultrasound to the target organ or gland.

In such embodiments, the one or more target organs or glands may be one or more of the salivary glands. Embodiments of the invention are not limited to a specific portion of the salivary gland. For example, in some embodiments, the target organ or gland may be the parotid gland, and in other embodiments, the target organ or gland may be the submandibular gland or sublingual gland. In still other embodiments, the target organ or gland may be a combination of the parotid, submandibular, and sublingual salivary glands or all of the parotid, submandibular, and sublingual salivary glands.

Without wishing to be bound by theory, the salivary glands may provide an excellent target for delivery of genetic material, and in particular, delivery of therapeutic genetic material which may direct synthesis of one or more beneficial transgenic protein. For example, salivary glands are encapsulated, which may limit diffusion of the genetic material beyond the target area. Salivary glands are also easily accessible and not essential to life. Therefore, salivary glands can be easily removed if safety concerns arise. Finally, protein synthesis in salivary glands is robust, and salivary glands are capable of releasing large quantities of a transgenic protein into the bloodstream of the subject.

Embodiments of the invention are not limited by the type of genetic material delivered. For example, in some embodiments, the genetic material may be a viral vector, and in other embodiments, the genetic material may be a non-viral vector. In still other embodiments, the genetic material may be siRNA. Without wishing to be bound by theory, the robust protein synthesis provided by salivary glands may allow therapeutic expression levels to be reached using a non-viral vector. Moreover, a non-viral vector may be administered to a salivary gland in multiple separate doses allowing therapeutic expression levels to be maintained over an extended period of time. Thus, treatment may be effectuated over an extend period of time using repeated dosing at regular intervals. For example, in some embodiments, a subject may be treated with a non-viral vector using ultrasound mediated delivery methods for greater than 1 year, and in other embodiments, the subject may be treated for greater than 5 years using such methods. In still other embodiments, treatment using a non-viral vector and ultrasound mediated delivery may be carried out at regular intervals throughout the lifetime of the subject. The intervals over which a non-viral vector may be administered by the methods of invention may vary. For example, in some embodiments, the subject may be administered a non-viral vector once per week, once every two weeks, or once per month, and in other embodiments, the method described herein may be carried out to deliver a non-viral vector to the subject once every 3-months, once every 6-months, or once per year.

In contrast, viral vectors are generally effective only after the first administration, and repeated administration is often not possible due to immune response, which precludes “re-dosing” of the viral vector. The ability to express a therapeutic protein from an exogenous vector after repeated administration, therefore, provides a significant improvement over the state of the art and should be considered surprising and unexpected.

In such embodiments, “genetic material” may include, DNA, RNA, DNA equivalents or RNA equivalents, which may include non-naturally occurring or modified nucleotides, or various hybrids including DNA nucleotides and/or RNA nucleotides and/or non-naturally or modified nucleotides, including, but not limited to siRNA, microRNA, antisense and the like. Additionally, the term “vector” may include any form of genetic material that can be actively translated once incorporated into a cell such as, for example, a plasmid. Examples of genetic material that may be delivered using the methods of embodiments of the invention include, but are not limited to, a primary RNA transcript that may be processed into messenger RNA and translated by a ribosome into a protein within the cytoplasm of the cell, or DNA that may be transcribed into a messenger RNA and translated into protein by a ribosome. Thus, the DNA vector may include various sequences required for its transcription and translation such as, for example, promoter and enhancer sequences. Additionally, RNA transcribed from a DNA vector and primary RNA transcripts may include, for example, introns that can be spliced following delivery, poly A sequences, other sequences required for the initiation and termination of its translation into protein, and the like.

In various embodiments, a vector may be delivered to a cell in order to produce a cellular change by, for example, expressing an exogenous gene or modifying expression of a naturally produced gene, and in particular embodiments, the vector may be delivered to illicit a therapeutic effect, or provide gene therapy. Therefore, in some embodiments, the vector may encode one or more whole or partial proteins that can be delivered either directly to the organism in vivo to produce a therapeutic effect by, for example, increasing or stimulating expression or exogenously expressing a necessary protein that is produced at insufficient levels or not produced at all by the subject. The whole or partial proteins expressed from such vectors may include, for example, anti-inflammatory cytokines, which may effect numerous inflammatory diseases and autoimmune diseases, a-Galactosidase A, which may effect treatment of Fabry disease, or additionally other lysosomal enzymes deficient in the family of lysosomal storages disease of which Fabry disease is a member such as, for example Tay-Sacs disease, Gaucher disease, Pompe disease, and the like. In addition, various antimicrobial or anti-biofilm peptides could be effective in dental medicine applications. In addition, various inflammatory or degenerative conditions of the salivary gland may be treated, including radiation-induced xerostomia and Sjogren's Syndrome.

Microbubble-enhanced ultrasound gene delivery was reported by Lawrie et al., Gene Ther., 7(23):2023-2027 (2000), which is hereby incorporated herein by reference in its entirety, and is well known in the art. For example, U.S. Pat. No. RE36,939 to Tachibana et al., which is hereby incorporated by reference in its entirety, describes microbubbles of gas in a liquid that are useful for the therapy of various diseases together with exposure to ultrasonic waves and U.S. Pat. No. 5,542,935 to Unger et al., which is hereby incorporated by reference in its entirety, discloses delivery gaseous precursor-filled liposomes that encapsulate a contrast agent or drug. All such microbubbles and methods for preparing microbubbles are encompassed by embodiments of the invention. In other embodiments, commercially available microbubbles such as, for example, DEFINITY® and OPTISON™, may be used in the methods described herein. In certain embodiments, microbubbles may be prepared by forming microspheres of a gas into a liquid. The gas, in such embodiments, may be any gas, and in particular embodiments, the gas may be physiological acceptable gas such as atmospheric air, oxygen, nitrogen, and the like or an inert gas such as noble gases, sulfur hexafluoride, perfluorocarbon gases, and the like. In other such embodiments, the gas may be a mixture of physiologically acceptable and inert gases. Similarly, the liquid of such embodiments, may be any liquid, and in particular embodiments, may be a physiologically acceptable solution such as, for example, saline solution, phosphate buffered saline (PBS), aqueous glucose solution, human serum albumin solution, and the like or combinations thereof at a physiologically acceptable pH and osmolarity. In embodiments in which the liquid is a physiologically acceptable solution, the solute, e.g., NaCl, glucose, or albumin, may be at any concentration. For example, in some embodiments, the solute may be about 1 wt. % to about 8 wt. % of the total solution, and in other embodiments, the solute may be about 3 wt. % to about 5 wt. % of the total solution. In particular embodiments, the solution may be PBS and may include 10 mM potassium phosphate having a pH of 7.4 in 0.9% NaCl solution.

In certain embodiments, microbubbles useful in embodiments of the invention may include a “shell” of, for example, albumin, lipids, phospholipids, metals, polymers, or combinations thereof, and in particular embodiments, the shell may be prepared from lipids or phospholipids. Numerous lipids may be used to produce microbubble shells such as those describes in WIPO Publication No. WO 2000/45856 and include, but are not limited to, fatty acids, phosphatides, glycolipids, glycosphingolipids, sphingolipids, aliphatic alcohols, aliphatic waxes, terpenes, sesquiterpenes, steroids, phosphocholines, phosphatidylcholines, phosphatidylethanolamines, phosphatidylserines, phosphatidylglycerols, and phosphatidylinositol, and some embodiments, 1,2-palmitoyl-sn-glycero-3-phosphocholine or 1,2-palmitoyl-sn-glycero-phosphatidylethanolamine, and in particular, L-1,2-palmitoyl-sn-glycero-3-phosphocholine and L-1,2-palmitoyl-sn-glycero-phosphatidylethanolamine. In yet other embodiments, microbubbles may be prepared from a biodegradable polymer and/or amphiphilic material such as collagen, gelatin, albumin, or globulin.

The size of the microbubbles useful in embodiments of the invention may vary based on their composition and/or the liquid or solution in which they are formed. For example, in some embodiments, the microbubbles may be from about 0.01 μm to about 100 μm in diameter. In other embodiments, the microbubbles may have a diameter of from about 0.1 μm to about 50 μm, and in still other embodiments, the microbubbles may have a diameter of from about 1 μm to about 10 μm or about 2 μm to about 4 μm.

The microbubbles of various embodiments may further include any number of additional materials such as, for example, an ultrasound contrast agent. Ultrasound contrast agents are known in the art and commercially available and include OPTISON™, as well as aqueous indocyanine green solution, agitated hypertonic saline, autoblood, aqueous maglumine diatriazoate, and the like.

In various embodiments, the genetic material may be either entrapped within the core or center of the microbubble or attached to an outer surface of the microbubble shell by chemical, electrostatic, or mechanical means. As such, microbubbles may be prepared in a solution that contains the genetic material meant for delivery, or microbubbles may be prepared in a first step and combined with the genetic material. In particular embodiments, the microbubbles may be prepared before being combined with the genetic material. For example, in some embodiments, genetic material may be added to a solution containing about 10 vol. % to about 25 vol. % microbubbles or about 10 vol. % to about 20 vol. % microbubbles, and in other embodiments, the genetic material may be combined with a solution containing about 15 vol. % microbubbles. In still other embodiments, genetic material may be combined with a solution containing about 12 vol. %, about 13 vol. %, about 14 vol. %, about 15 vol. %, about 16 vol. %, about 17 vol. %, about 18 vol. %, about 19 vol. %, about 20 vol. %, about 22 vol. %, or about 25 vol. % microbubbles.

The amount of genetic material combined with the prepared microbubbles may vary among embodiments. For example, in some embodiments, from about 1 μg to 100 μg of genetic material, and in other embodiments, that amount of genetic material may be from about 5 μg to about 90 μg, from about 10 μg to about 75 μg, from about 15 μg to about 50 μg. As such, the amount of genetic material in the microbubble solution may vary. For example, in various embodiments, the amount of genetic material in genetic material/microbubble solution may be from about 0.01 μg/μl to about 10 μg/μl, about 0.1 μg/μl to about 5 μg/μl, and in certain embodiments, about 1 μg/μl to about 2.5 μg/μl. In particular embodiments, the amount of genetic material in the genetic material/microbubble solution may be about 0.5 μg/μl, about 0.75 μg/μl, about 1 μg/μl, about 1.5 μg/μl, about 2 μg/μl, about 2.5 μg/μl, about 3 μg/μl, about 4 μg/μl, or about 5 μg/μl.

In some embodiments, the solution prepared as described above may be directly administered to the target organ or tissue, and in other embodiments, the concentration of genetic material entrapped within microbubbles or attached to microbubbles in the solution prepared as above may be decreased by adding additional solution or increased by removing a portion of the liquid. In still other embodiments, microbubbles prepared as described above may be dried to form a dry powder, and the dried powder may be rehydrated by for example adding saline, PBS, or an aqueous glucose solution to the powder. Thus, the concentration of various microbubble containing solutions may vary indefinitely.

The step of contacting may take place by any method. For example, in some embodiments, a solution of microbubbles and entrapped or attached genetic material may be injected directly into the target salivary gland. In other embodiments, a solution of microbubbles and entrapped or attached genetic material may be injected into a vein or artery that will carry the microbubbles to the target salivary gland, and in still other embodiments, a solution of microbubbles and entrapped or attached genetic material may be washed over the target salivary gland as part of, for example, lavage. In yet other embodiments, the microbubbles and entrapped or attached genetic material may be infused into the salivary glands. For example, in some embodiments, a catheter may be used to infuse the microbubble solution into salivary gland.

Without wishing to be bound by theory, the amount or concentration of microbubbles and entrapped or attached genetic material may vary depending on the mode of administration to the target salivary gland used. In addition, the skilled artisan may consider other factors that may limit the efficiency of delivery of the genetic material to the target organ or gland. For example, DNA attached to the microbubble can be neutralized by circulating deoxyribonucleases (DNases), or the microbubble may be degraded by, for example, proteases, lipases, carbohydrate-cleaving enzymes, and other degradation pathways. In other cases, free DNA released from the microbubble inside the target organ may not enter a cell or nucleus where it can be transcribed, or part of a microbubble shell may remain attached to a DNA molecule and prevent transcription. Accordingly, the amount of microbubbles and entrapped or attached genetic material may be from, for example, about 1×10⁶ bubbles/ml of microbubbles to about 1×10¹¹ bubbles/ml of microbubbles and, in particular embodiments, about 1×10¹⁰ bubbles/ml of microbubbles.

In some embodiments in which microbubbles and entrapped or attached genetic material may be injected into the target organ or gland at a pressure achieved by administering about 1 ml to about 500 ml of the microbubble solution in about 2 seconds to 10 seconds. In other embodiments, the injection pressure may be increased by a method such as, for example, occlusions or as a function of an injector device such as an infusion pump. However, the pressure should be below that which might damage the target salivary gland.

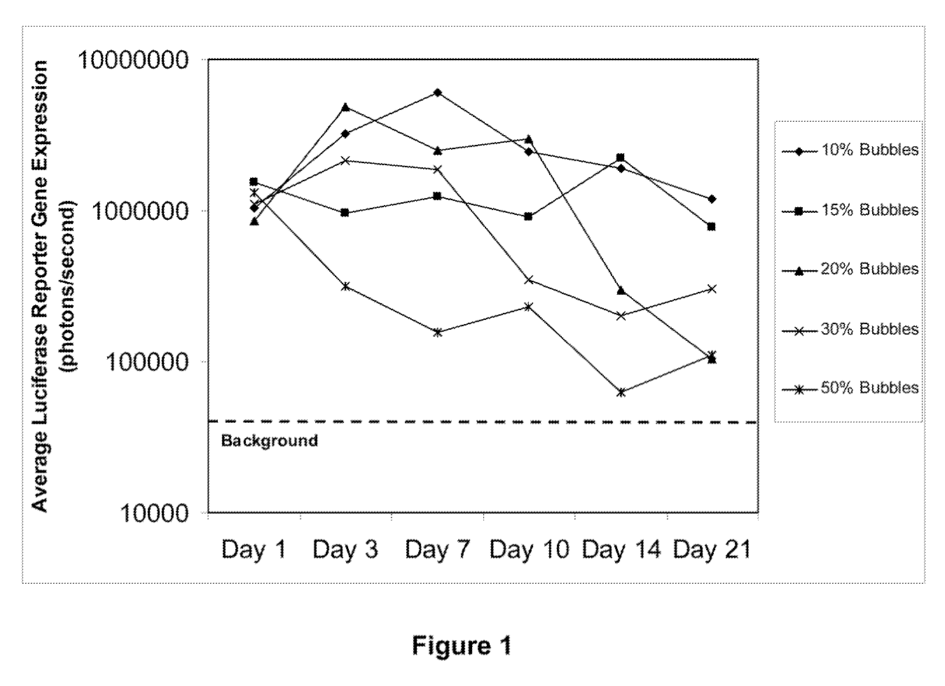

The solution used to administer the microbubbles may contain any concentration of microbubbles. For example, in some embodiments, the solution may be up to about 50 vol. % microbubbles, and in other embodiments, the solution may be up to about 30 vol. %, up to about 20 vol. %, up to about 15 vol. %, up to about 10 vol. %, or up to about 5 vol. % microbubbles or less than about 5 vol. % microbubbles. In particular embodiments, the microbubble solution may be from about 5 vol. % to about 50 vol. % microbubbles, and in still other embodiments, the microbubble solution may be from about 5 vol. % to about 30 vol. % microbubbles, about 10 vol. % to about 20 vol. % microbubbles, or about 10 vol. % to about 15 vol. % microbubbles. Without wishing to be bound by theory, administration of an effective amount of microbubbles may be more readily facilitated by administering a microbubble solution that is less than 30 vol. % or less than 20 vol. % microbubbles. For example, as illustrated in FIG. 1, Luciferase reporter gene expression may be more robust when the reporter gene is administered to salivary glands by delivering a solution containing less than 20 vol. % microbubbles, and in particular, by delivering a solution containing 15 vol. % or 10 vol. % microbubbles, even though a solution including up to 50 vol. % microbubbles provides sufficient delivery to produce Luciferase expression over background levels.

A wide variety of ultrasound equipment and methods, frequencies, modes, energies, and the like may be used within embodiments of the invention. Ultrasound energy may cause gas-filled microbubbles to resonate or burst into smaller fragments and may induce bubble resonance that physically deforms or disrupts cell membranes of the cells of the target tissue or organ causing, for example, cavitation, microstreaming, and perforating or increasing the permeability of cell membranes. Bubble resonance is typically described as sonoporation or non-inertial cavitation, while the bubble destruction is described as inertial cavitation. Without wishing to be bound by theory, in embodiments of the invention bursting of microbubbles and/or deformation of cell membranes as a result of applying ultrasound energy to the target salivary gland may allow the encapsulate or attached genetic material of the microbubble to enter cells of the target organ or gland where they may elicit a therapeutic effect.

The term “ultrasound” generally refers to a form of energy consisting of mechanical vibrations having frequencies that are above the range of human hearing with a lower frequency limit of about 20 kHz. As used herein, the term “ultrasound” may include diagnostic, therapeutic and focused ultrasound, any of which may be utilized in embodiments, of the invention. Diagnostic ultrasound generally employs frequencies of about 1 MHz to about 15 MHz and uses an FDA recommended ultrasound energy source of up to about 100 mW/cm². In contrast, therapeutic ultrasound uses an WHO recommended ultrasound energy source in a range up to about 3 mW/cm² to about 4 W/cm.sup². Focused ultrasound (FUS) and high intensity focused ultrasound (HIFU) allows thermal energy to be delivered without an invasive probe, see Morocz et al., 1998 Journal of Magnetic Resonance Imaging 8(1):136-142 (1998), Moussatov et al., Ultrasonics, 36(8):893-900 (1998), and TranHuuHue et al., Acustica, 83(6):1103-1106 (1997), each of which are hereby incorporated by reference in their entireties. In various embodiments of the invention, diagnostic, therapeutic, FUS, HIFU or combinations thereof may be used to induce sonoporation, inertial cavitation, non-inertial cavitation, and/or acoustic activation.

“Ultrasound disruption threshold” refers to the ultrasound energy required to destroy microbubbles and induce sonoporation, inertial cavitation, non-inertial cavitation, and/or acoustic activation. Ultrasound energy is typically described as a combination of parameters including intensity, which may be defined in terms of mechanical index, pressure, decibels, or energy per surface area, the ultrasound frequency, pulse mode, pulse repetition frequency, pulse duration, and other parameters. In embodiments of the invention, ultrasound may be applied to a target organ or gland with sufficient strength to cause the microbubbles to resonate and/or burst and disrupt the cell membranes of cells within the target tissue or organ without permanently damaging the target tissue or effecting surrounding tissues. As used herein, any transient permeabilization or disruption of the cell membrane by the ultrasound energy source is not encompassed by the term “damage” or “damaging”.

The various parameters of the ultrasound energy source used may vary among embodiments of the invention, and the skilled artisan may vary each parameter as necessary to improve the effectiveness of treatment. For example, in some embodiments, the acoustic power density of the ultrasound energy source may from about 0.5 W/cm² to about 100 W/cm², and in other embodiments, the power density of the ultrasound energy source may be from about 1 W/cm² to about 15 W/cm². In some embodiments, the frequency of the ultrasound energy source may be from about 0.015 MHz to about 10.0 MHz, and in other embodiments the frequency of the ultrasound energy source may be from about 0.02 MHz to about 5.0 Mhz. In certain embodiments, the frequency of the ultrasound energy source may be from about 0.5 MHz to about 3 Mhz. Similarly, the duty cycle may vary. For example, in some embodiments, ultrasound may be carried out at a 50% duty cycle in 4×30 s bursts.

The period of exposure may vary among embodiments and may be adjusted based on the observations of the skilled artisan to effectuate treatment. For example, in some embodiments, the period of exposure may be from about 10 milliseconds to about 60 minutes, and in other embodiments, the period of exposure may be from about 1 second to about 20 minutes. In still other embodiments, the period of exposure may be from about 10 seconds to about 10 minutes or from about 30 seconds to about 3 minutes.

As indicated above, numerous courses of treatment can be derived from the parameters described above. Thus, the following non-limiting examples of courses of treatment are provided for clarity. In some exemplary embodiments, a patient may be exposed to an ultrasound energy source at an acoustic power density of from about 0.05 W/cm² to about 10 W/cm² with a frequency ranging from about 0.015 MHz to about 10 MHz for a period of from 30 seconds to about 2 minutes, and in other exemplary embodiments, a patient may be exposed to an ultrasound energy source at an acoustic power density of greater than about 100 W/cm² for reduced periods of time such as, for example, 1000 W/cm² for a time period of 30 milliseconds or less.

In certain embodiments, the methods described throughout may be used to treat pulmonary arterial hypertension (PAH). PAH is a rare, deadly, and incurable disease with a mean survival of 2.8 years from onset of symptoms if left untreated characterized by extensive narrowing of the pulmonary vasculature leading to progressive increases in pulmonary vascular resistance, right ventricular failure and death. PAH can be further classified into either: 1) an “associated” form where there is an identifiable cause for the pulmonary vascular changes (connective tissue diseases, congenital heart diseases, HIV, cirrhosis with portal hypertension), or 2) two related diseases known as Familial PAH (FPAH) in which the disease is related to mutations in the BMPR2 gene and Idiopathic PAH (IPAH), where no identifiable cause exists. PAH in general is a rapidly progressive and fatal disease and this is particularly true of IPAH which has survival rates at 1, 3, and 5 years of 68%, 48%, and 34%, respectively with an average survival from onset of symptoms of 2.8 years if left untreated. There are currently three classes of approved therapeutics for treating PAH, endothelin receptor antagonists, phosphodiesterase inhibitors, and prostacyclins. Of these, prostacyclin appears to be the most effective therapy; however, complicated delivery systems and potential side effects associated with the present formulation of prostanoids (e.g. prostacyclin) have deterred some patients and caregivers from instituting this highly effective class of agents.

Prostacyclins, endothelin receptor antagonists and phosphodiesterase inhibitors have been evaluated in PAH patients and have demonstrated improvements in symptoms, exercise tolerance, and in hemodynamics, over the short term, but intravenous prostacyclins are the only class of therapeutic shown to improve survival in PAH in clinical trials. However, serious complications, including cardiogenic shock and death are associated with inadvertent infusion interruptions with the IV prostacyclin, epoprostenol, due to its short half-life (approximately 6 minutes). In addition, because intravenous preparations require an indwelling central venous catheter, life threatening blood stream infections have emerged as a limiting factor for this form of administration. Thus, alternative routes of delivery of prostacyclin are being investigated using stable prostacyclin analogues administered orally (beraprost), subcutaneously (treprostinil), or by inhalation (Iloprost). Unfortunately, severe site pain limits the use of subcutaneous treprostinil, and the current inhaled route is hampered by the requirement for multiple timely inhalations (6-9, 10-15 minute inhalations) during the day. In addition, current oral preparations are not clinically efficacious and cause intolerable gastrointestinal side effects. Thus, the complicated delivery systems and potential side effects associated with the present formulation of prostanoids have deterred some patients and caregivers from instituting this highly effective class of agents. In light of this, a compelling need exists for alternative delivery systems that provide sustained, effective, and convenient dosing for patients.

In some embodiments, PAH may be treated by delivering an anti-inflammatory cytokine, such as for example, a cox-1 or cox-2 or a cox-1/PGIS fusion protein transgene to salivary glands using the methods described above which may result in in vivo production of prostacyclin. In other embodiments, genetic material may delivered to the salivary glands that would result in the production of prostacyclin in vivo such as, for example, genetic material encoding prostacyclin, an active portion of prostacyclin, or a prostacyclin analog. In still other embodiments, the genetic material may be a fusion protein of cox-1 and PGIS. Such embodiments may allow for endogenous production of prostacyclin within the patient's own body, throughout the entire lifetime of the patient. Gene therapy has shown that prostacyclin can be endogenously produced through expression of the enzyme prostacyclin synthase (PGIS), thereby providing proof-of-principle of the efficacy of gene therapy for treatment of PAH in animal models.

In some embodiments, methods for delivering genetic material to treat PAH may utilize gene transfer technology such as, for example, viral gene delivery vectors or other types of gene delivery technology. In particular, in certain embodiments, non-viral DNA vectors may be delivered to cells using the ultrasound induced microbubble cavitation as described above. Without wishing to be bound by theory, in such embodiments, the non-viral DNA vector may be delivered while evading host immune responses, allowing for re-dosing of the PGIS transgene and providing periodic boosters throughout the lifespan of the patient. In some such embodiments, the salivary glands may be used as the therapeutic biosynthesis site. In particular embodiments, a Cox-1/PGIS fusion protein may be delivered to the patient using ultrasound induced microbubble cavitation. In such embodiments, dramatically higher levels of prostacyclin than PGIS alone may be produced providing superior levels of prostacyclin therapeutic. The extremely short half-life of prostacyclin makes the anatomical site of production a consideration. The venous drainages of the parotid and submandibular salivary glands are the posterior and anterior facial veins, respectively. These drain into the external and internal jugular veins respectively and then directly into the superior vena cava and the right heart. Thus, the salivary glands are especially proximal to the anatomical target of the therapy.

In other embodiments, prostacyclin (PGI2), an endogenous substance produced by vascular endothelium, may be delivered using ultrasound induced microbubble cavitation. PGI2 has potent vasodilatory, antiplatelet and antiproliferative properties; however, development of oral PGI2 formulations has revealed that PGI2 can have deleterious gastrointestinal effects when ingested. Moreover, prostacyclin has rarely been measured in human saliva, and only its breakdown product 6-keto-prostaglandin F1a(6-K-P) has been detected, suggesting that PGI2 is rapidly degraded in the proteolytic environment of the saliva. Therefore, while we cannot predict a priori what proportion of the PGI2 gene therapeutic will distribute to the saliva versus the blood, we can be confident that any PGI2 entering the saliva will be substantially degraded before it reaches the stomach.

Advantages of the embodiments of the invention include a site for PGI2 biosynthesis that allows for re-dosable therapy that can be targeted to the salivary glands derives from the relative simplicity of salivary duct catheterization, which can be performed in humans as an outpatient procedure such as a sialogram. Additionally, viral vector systems have a limited duration of expression, including varying degrees of immunogenicity. Thus far, even “integrating” viral vector systems have not been able to enact permanent gene replacement, as therapeutic transgenes persisting in the genome are eventually silenced by intracellular mechanisms that are incompletely understood. Most unfortunately, all viral vector systems are fundamentally limited by dramatic increases in the host immune response with each successive dose. The corresponding challenge is that re-dosing of a gene therapy with currently available viral vector systems is impracticable. Enacting a redosable gene therapy strategy for PAH must, therefore, overcome the basic mechanism of host immune response to the vector.

Extracellular and intracellular immune reactions against viral based vectors have been reported against both viral capsid proteins and viral nucleic acid sequences. The relative contributions of viral capsids and viral genomes to the host immune response in gene therapy have not been firmly established, although the source of most extracellular immune response (mainly T-cell mediated) appears to be attributable to viral capsid antigens being presented on MHC Class I receptors. Numerous attempts to modify viral capsids to make them less inflammatory have thus far shown limited translational utility. In light of these considerations, the re-dosable gene delivery vector may be a non-encapsidated DNA “minicircle” containing a circularized expression cassette without inverted terminal repeats or CpG sequences of viral or bacterial origin. Such vectors have been developed but the challenge to in vivo application of this new technology in gene therapy is delivery to the target cell. Cell membranes are highly resistant to foreign DNA, and lipofection techniques that function well in vitro have been disappointing when applied to in vivo gene transfer. Similarly, various attempts to mimic viral methods of cell entry with various DNA-associated “nanoparticles” have not yet shown unequivocal success.

The ultrasound-assisted gene transfer (UAGT) technique utilized in the embodiments of the invention approaches the problem of non-viral gene delivery in an entirely novel and innovative manner. UAGT physically disrupts the target cell membrane by a phenomenon described as “sonoporation”. In this technique, microbubbles can be targeted with high mechanical index ultrasound at a frequency at which they are resonant. This energy destroys the bubbles, leading to local cavitation, which transiently disrupts the cell membrane, allowing entry of a plasmid vector. While UAGT's practicality for solid organ transfer in vivo is still unclear, its utility and practicality for gene transfer to the epithelial tissues of the salivary gland is now evident. Coupling this gene transfer technique with an appropriate gene payload (e.g. prostacyclin synthase) and applying it as a therapeutic strategy for PAH may effectively treat this disease.

The methods provided in embodiments of the invention offer substantial improvements over the state of the art. For example, introduction of a vector containing a gene therapeutic protein into one or more salivary glands may result in robust expression of the therapeutic protein, which may be improved over expression in other organs. Additionally, genetic material may be injected directly into the salivary glands and salivary glands are encapsulated providing easy access and an improved means for delivery over systemic administration. In fact, in some embodiments, greater than 20% of a vector directly administered to the salivary glands may effectively transfect the cell using the ultrasound mediated methods described hereinabove. Such results represent a substantial improvement over the prior art and should be considered surprising and unexpected. Moreover, salivary glands are not essential to life, and if exogenous expression of a therapeutic protein becomes unnecessary or dangerous, can be removed without effecting the overall health of the subject.

In various embodiments, the methods described may be used alone or in combination with other forms of therapy including, for example, chemotherapy, hormone replacement therapy, enzyme replacement therapy, vasoactive peptide therapy, dental anti-biofilm or antimicrobial peptide therapy and the like.

### EXAMPLES

Although the present invention has been described in considerable detail with reference to certain preferred embodiments thereof, other versions are possible. Therefore the spirit and scope of the appended claims should not be limited to the description and the preferred versions contained within this specification. Various aspects of the present invention will be illustrated with reference to the following non-limiting examples. The following examples are for illustrative purposes only and are not to be construed as limiting the invention in any manner.

### Example 1

The salivary glands of mice were infused with 50 μl of a solution containing 15% microbubbles and 50 μL of a non-viral vector including the firefly Luciferase gene under the control of the CMV promoter using a catheter. Following infusion, a SoniGene (VisualSonics, Inc) ultrasound was used to deliver ultrasound energy at 2 mW/cm² at a frequency of 1 MHz, 50% duty cycle at 4×30 s pulses.

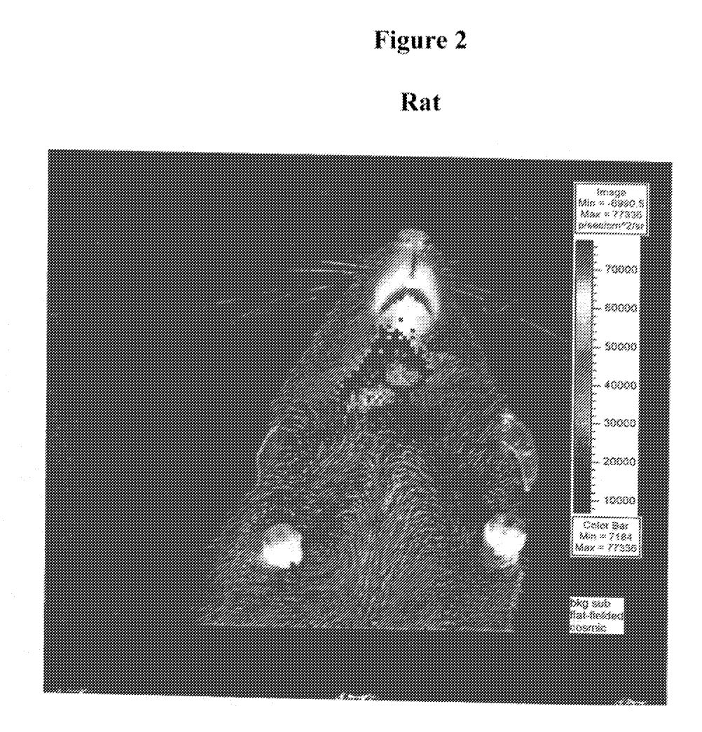

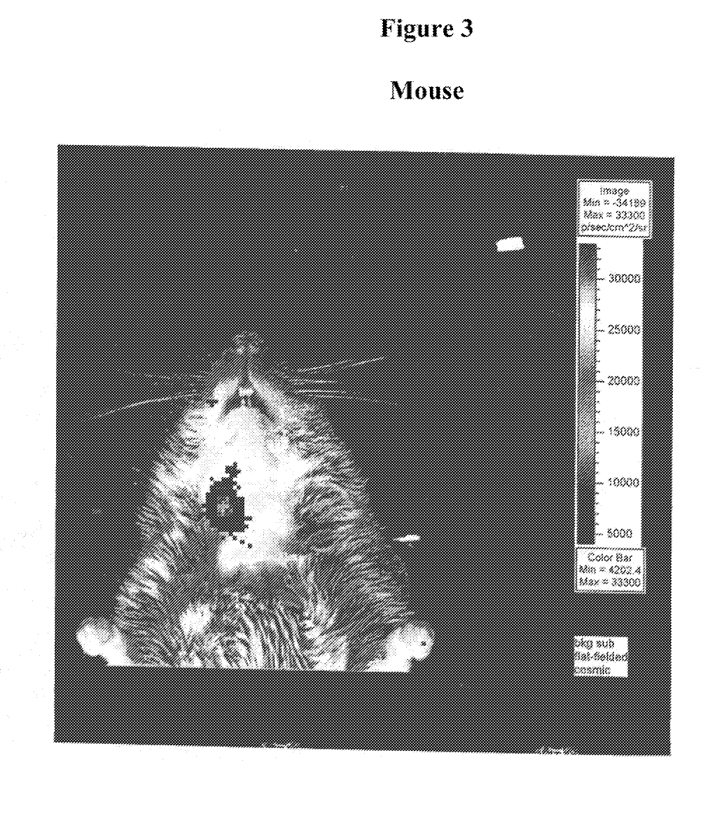

Expression of Luciferase was monitored using a CCD camera. Exemplary results are shown in FIGS. 2 and 3.

### Example 2

The ultrasound-assisted gene delivery strategy presented in the embodiments herein meets the criteria of a practicable, re-dosable gene transfer strategy for delivering Cox-1/PGIS to the salivary glands. The salivary glands may be exceptionally well suited to endogenous production of biotherapeutics targeted to the intravascular space. Thus, to achieve our goal of re-dosable, endogenous production and endocrine secretion of PGI2, we leverage the recent development of several new gene therapy technologies that appear to be enabling of such a goal. The cornerstone innovation of this proposal will be the aggregation of these new technologies into a single in vivo gene therapy treatment paradigm in which the metrics are physiological and closely mimic endpoints that would be utilized in a human clinical trial.

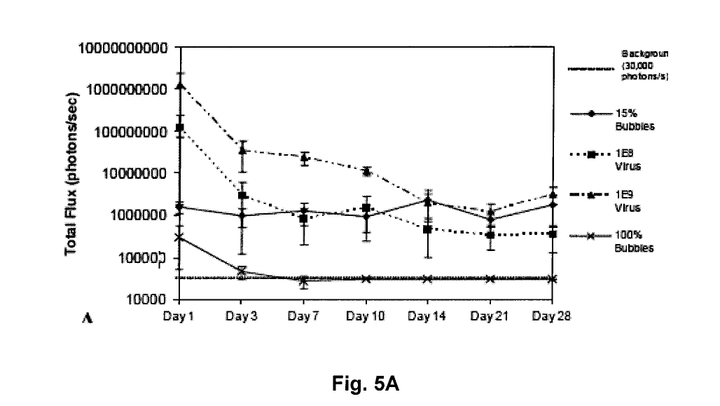

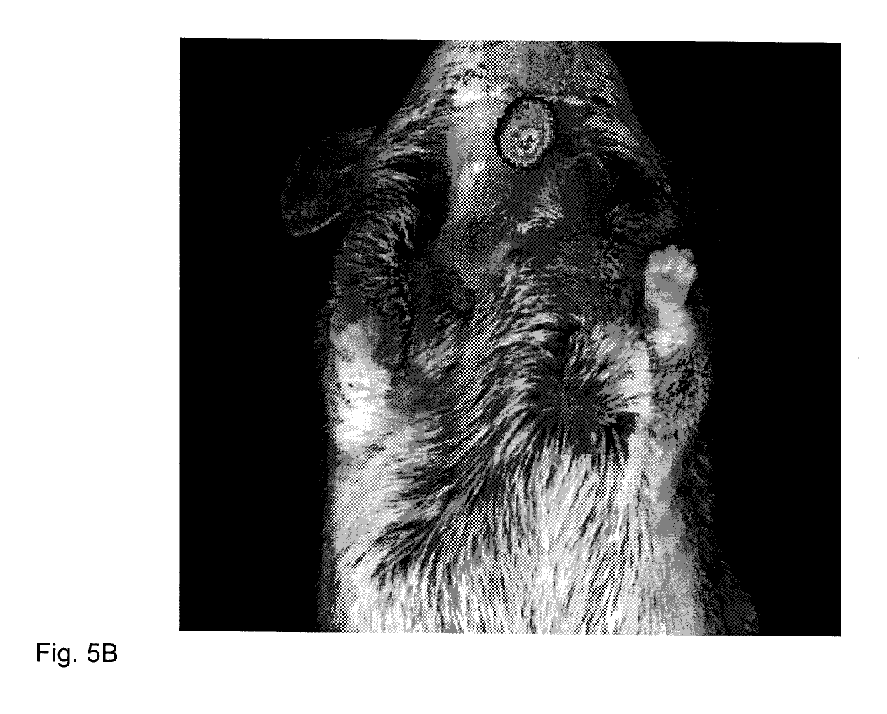

Delivery of the Cox-1/PGIS fusion protein to the submandibular gland of the rat using UAGT and measure serum levels of PGI2 on days 2, 7, and 21 post-treatment. FIG. 5A shows the total flux at various timepoints relative to Adenoviral gene delivery providing gene expression over a 28 day period using UAGT compared to an Adenoviral vector. Background was observed to average ˜30,000 photons/sec. FIG. 5B shows pseudocolored image of photon intensity overlayed on a photograph at 14 days post-UAGT indicating the spatial localization of gene expression to the salivary gland in situ

One consideration of both immediate and long-term importance is whether UAGT can achieve the gene expression levels necessary to produce therapeutically meaningful levels of PGI2. Potential translation of this technology from mice to humans is speculative, but two considerations lead us to be optimistic. First, our data have demonstrated that we can achieve gene expression levels using UAGT roughly equivalent to 1×10⁷-1×10⁶ viral particles of an adenoviral vector (see FIG. 5A). A salivary gene therapy clinical trial currently underway is utilizing a dose escalation protocol encompassing five steps from 4.8×10⁷ to 3.5×10¹⁰ viral particles in humans. Thus, our expression level is well within the range thought to be biologically meaningful in humans, particularly when one considers the mouse-to-human scaling factor. Second, a recent study showed steady state levels of ˜2.5 ng/mL of human parathyroid hormone (hPTH) in the serum of mice after delivery of hPTH with 1×10¹⁰ viral particles of an adenoviral vector. The average dosage of epoprostenol used in PAH in humans is ˜10-40 ng/kg/min (or ˜10-40 μg/mL/min). Thus, when we consider that nanogram/mL levels of transgene can be achieved with adenoviral systems, even without the enzymatic amplification effect our system will leverage (since each Cox-1/PGIS transgene molecule delivered will produce large numbers of PGI2 therapeutic molecules), nanogram/kg/mL serum levels may be achievable with reasonable dosages.

The highly efficient production of PGI2 by the Cox-1/PGIS fusion protein construct has been reported, and the utility of the UAGT system for achieving robust and persistent transgene expression in the salivary gland has been documented (see e.g., FIG. 5). With these two key feasibilities established, Cox-1/PGIS in the salivary glands of a living animal may result in some portion of the PGI2 produced following this intervention entering the bloodstream that can be measured.

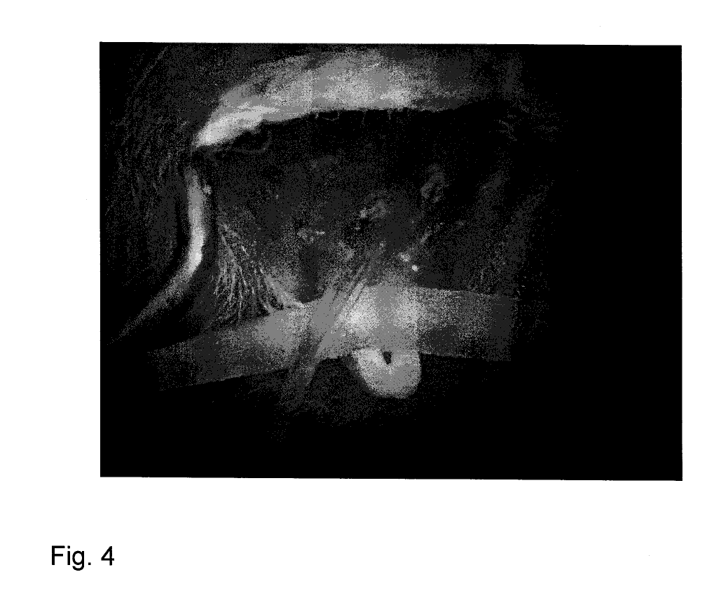

Cox-1/PGIS gene transfer to the rat salivary gland will be carried out as follows: About 9×10⁷ microbubbles and 50 μg of plasmid DNA will be delivered to both the right and left submandibular salivary glands of rat subjects. Briefly, animals will be anesthetized with a Ketamine/Xylazine cocktail and placed in a frame that holds the mouth open. The tongue will be retracted to expose the opening of the submandibular ducts, and a fine plastic catheter will be placed in the duct and advanced ˜1 cm then held in place with Superglue (see FIG. 4). 50 μl of plasmid/microbubble solution will be injected into the gland and perfusion of the gland will be visualized with ultrasound. The previously depilated skin overlying the salivary gland will be covered with a layer of commercial ultrasound gel. A SoniGene (VisualSonics, Inc., Toronto, Canada) probe will be lowered to lightly contact the skin and 4×30 second bursts will be applied at 2.4 W/cm2, 50% duty cycle with a 10 second interval between bursts. The SoniGene probe will then be retracted and the animal returned to its cage.

In order to non-invasively monitor transgene expression and correlate relative expression levels with resultant PGI2 production for interanimal comparisons, Cox-1/PGIS fusion protein will be monitored as the first gene in a bicistronic expression cassette. This cassette will be driven by a cytomegalovirus (CMV) promoter upstream of the Cox-1/PGIS protein ending in a stop codon; downstream of the stop codon will be an internal ribosomal entry site (IRES) sequence upstream of a Luciferase reporter gene. This design will result in the serial expression of both distinct transgenes, with the Luc reporter being expressed at ˜ 1/10 of the level of the therapeutic transgene. Level of Luc expression can be monitored as a direct surrogate of Cox-1/PGIS expression and by using the Xenogen IVIS system the total photon flux as a reproducible metric of transgene expression will be measured (see FIG. 5). The ratio of Luc expression to PGI2 produced (see below) will be compared between animals, with any discrepancies noted for future investigations.

Dose escalation by serial treatments will be carried out as follows: Animals in a serial treatment group (n=8) will undergo the same UAGT procedure as described above but the treatment will be repeated every 5 days for three treatments, total. As illustrated above, we will be able to obtain semiquantitative measurements of transgene activity through non-invasive Luc imaging. Therefore, we can compare the magnitude of transgene expression between the single treatment and serial treatment groups over the duration of the treatment course.

Detection and relative quantitation of 6-keto-PGF1α. For detection and relative quantitation of PGI2 levels, we will utilize the one-step immunoassay method, which detects the stable metabolite of PGI2, 6-keto-PGF1α (6-K-P). This kit-based assay will allow for direct analysis of plasma without any pretreatment of the samples. Because 6-K-P is stable, inter-sample variability due to handling or storage conditions is minimized Results will be confirmed with liquid-chromatography tandem mass spectrometry as previously described for direct detection of prostacyclin. Absolute levels of prostacyclin will be determined using isotope-dilution mass spectrometry.

### Example 3

Delivery of Cox-1/PGIS fusion protein as a treatment of PAH.

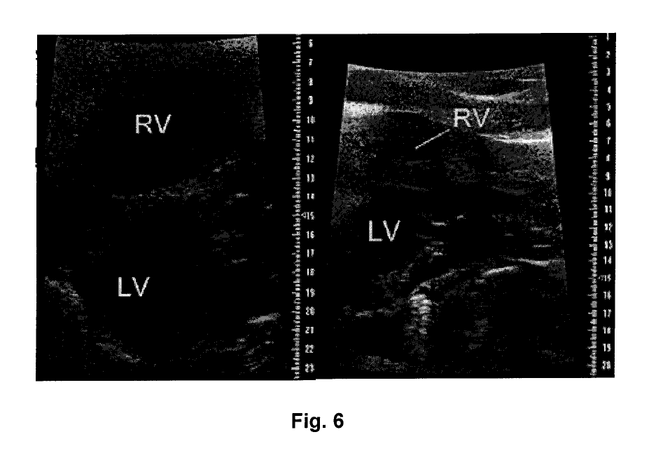

Echocardiographic evidence of severe PAH in a left-pneumonectomy/MCT rat model will be replicated and expanded with the use of high frequency echocardiography. This model produces both physiologic findings (systemic pulmonary pressures) and pathologic lesions (plexiform lesions and vascular remodeling) in the lung similar to the human condition, as well as the changes in the right ventricle (hypertrophy, enlargement and dysfunction) indicative of severe end-stage clinical PAH within 8-11 days of MCT administration. The degree of RV enlargement is closely linked to survival in humans patients with PAH. The ability to accurately assess the RV in this proposal, therefore, will provide added validity and applicability of this model to the human condition. This model has proven reliable, with 100% of animals developing echocardiographic evidence of pulmonary hypertension and right ventricular enlargement. FIG. 6 shows comparative images of the right ventricle of a rat from our lab with advanced PAH relative to a normal control. Note the significant increase in size of the right ventricle indicative of severe pressure overload from advanced pulmonary hypertension.

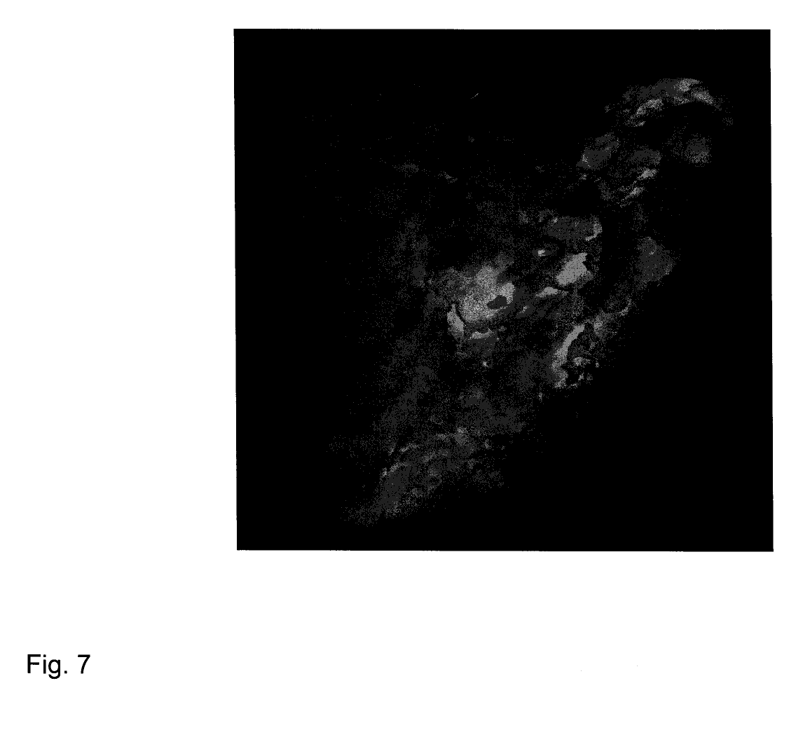

Quantifiable, 3-D modeling of pulmonary vasculature with Micro-CT will be provided by perimortem perfusion of the pulmonary circulation of the rat with MicroFil, a liquid contrast agent which polymerizes within minutes, results in a X-ray opaque cast of the intravascular compartment of the pulmonary circulation. This cast can then be scanned with ex vivo micro computed tomography and reconstructed in three dimensions with resolutions ˜10 μm, allowing for extremely detailed and quantitative analysis of the precapillary vascular changes that occur during the development of pulmonary hypertension. This technique is demonstrated in FIG. 7 using an ex vivo MicroCT scanner to build a 3-D model of the precapillary pulmonary circulation in one lobe of the right lung of a normal rat. These reconstructions can subsequently be analyzed to determine: 1) total intravascular volume within a unit volume, 2) mean vessel diameter within a unit volume and 3) percent contribution of various ranges of vessel caliber to the total in (1) to give information about whether particular sizes of vessels are selectively lost in PAH. As has been previously shown, this technique is sensitive to and descriptive of the vascular pruning that occurs in PAH.

24 Male Sprague-Dawley rats weighing less than 200 g will undergo a left pneumonectomy (Day 1). One week later (day 8), these rats will receive 60 mg/kg MCT as a subcutaneous injection. These two interventions should result in consistent development of PAH and right ventricular dilatation within 2 weeks following MCT. Animals that die prior to MCT injection will be replaced.

Eleven days after MCT (Day 19), animals (n=12), will be treated with Cox-1/PGIS gene transfer to both salivary glands. Twelve (12) additional, untreated animals will serve as PAH controls, and two animals will be treated with an irrelevant plasmid (CMV-Luciferase). Treatments will be administered as described above; however if dose escalation by serial treatments results in substantially more PGI2 production, a third group of animals (n=12) will be added wherein treatment is given on days 19, 24 and 29.

All animals surviving will be sacrificed on Day 36. Untreated PAH rats meet humane criteria for euthanasia an average of 28 days post administration of MCT, and >⅔ of untreated animals meet such criteria no later than 35 days post-MCT. The following treatment related endpoints will be assessed and differences between treatment groups analyzed using SPSS® stat software (p values <0.05, considered significant).

1. Echocardiographic analysis of right heart and pulmonary artery changes: Echos will be performed to assess pulmonary pressures and assess RV and pulmonary artery (PA) morphology prepneumonectomy, pre-MCT, weekly after MCT administration and at time of sacrifice. In addition to the obvious morphological observations made during the development of PAH in this rat model shown in FIG. 7, we have developed a full echo exam protocol to assess the full impact of PAH on the rodent heart. FIG. 7: High frequency echocardiography of PAH (A) and normal (B) animals. RV=Right Ventricle; LV=Left Ventricle.

These advanced measurements will not only provide a rough approximation of PA pressures but also evaluate main pulmonary artery morphology and left and right ventricular interactions, which are critical for determining survival in the human condition.

2. Hemodynamic Assessment: On the day of sacrifice (Day 36), animals will have a Millar catheter inserted into the internal jugular vein and advanced under ultrasound guidance into the PA. Right atrial pressure, PA pressures, cardiac output and pulmonary vascular resistance will be determined, after which point the animal will be sacrificed and lungs and heart processed either for MicroCT or histological analysis (see below).

3. Analysis of precapillary pulmonary vascular architecture using Micro CT: Precapillary vascular pruning has emerged as one of the most dramatic experimental measurements available for evaluating therapeutics in rodent models of PAH. We will perform these measurements as a terminal procedure in animals on Day 36 or on those animals meeting humane criterion for euthanasia only after echo and hemodynamic evaluation. The animal is given a deep, fatal dose of Ketamine/Xylazine mixture and the chest is opened to expose the heart and great vessels. An incision is made in the right ventricle and a catheter is advanced into the pulmonary artery and secured with surgical silk. The pulmonary circulation is then perfused with 20 mL of heparinized saline and the left ventricle is cut to allow the perfusate to escape without entering the systemic circulation. 14.7 mL of MicroFil (0.7 ml curing agent/7 ml diluent) is then infused at a rate of 2 ml/min. Afterward, the heart and the remaining lung are removed in one piece, and further separated by cutting the pulmonary artery and veins. The lung is wrapped in plastic wrap and sits overnight at room temperature to allow the MicroFil to cure. The following morning, the lung is transferred to 10% formalin for 24 hours before being scanned. Lungs are scanned in a Skyscan 1172 ex vivo MicroCT scanner by placing them in a Styrofoam frame and mounting on a rotating stage 12 cm from the X-ray source. Datasets are reconstructed in 3 dimensions using a networked computer cluster using NRecon Software and analyzed with CTAn and CTVol programs (all software provided by SkyScan, Kontich, Belgium). 3-D vascular reconstructions are analyzed according to the following parameters: 1) total intravascular space within a unit volume, 2) mean vessel diameter within a unit volume and 3) percent contribution of various ranges of vessel caliber to the total.

Histological Analysis: The lungs and hearts of 4 animals from each of the treatment groups and two naïve animals will be analyzed using histology. At the time of sacrifice, animals will be perfused systemically via the aorta with heparinized saline followed by 4% paraformaldehyde. Lungs will be removed and processed for paraffin embedding. 5 μm sections will be cut and stained using a combined elastin and modified trichrome stain as previously described. Arterial density and morphometric analysis (intimal area, medial areas and percentage of luminal occlusion) of arteries 15 to 50 μm in diameter will evaluated using a computer-based Bioquant II Morphometric System. Hearts will be also be removed and the LV and RV will be separated (septum remains with RV) and weighed individually to obtain RV/LV ratios. These ratios, a reflection of RV mass, are indicative of the downstream effects of pulmonary hypertension on the heart and provide supplemental information on the effect of our treatment regiments on the disease.

### Example 4

Delivery of Aquaporin-1 to the salivary glands of humans with xerostomia.

The treatment of most head and neck cancer patients includes ionizing radiation (IR). Salivary glands in the IR field suffer irreversible damage. There is no conventional treatment available to correct this condition. A recombinant human aquaporin-1 (hAQP1) vector will be developed to safely transfer the hAQP1 cDNA to parotid glands of adult patients with IR-induced salivary hypofunction, resulting in an elevated salivary output. hAQP1, the archetypal water channel, is a plasma membrane protein that facilitates water movement across lipid bilayers. Rat and minipig studies have clearly shown that the hAQP1 strategy for restoring salivary flow to IR-damaged salivary glands is effective. The purpose of this clinical protocol will be to test the safety of a hAQP1 vector, with some measures of efficacy, in adult patients with established IR-induced parotid gland hypofunction. The targeted tissue site for the hAQP1 vector in the proposed study will be a single parotid gland. In this Phase 1 dose-escalation study, safety will be evaluated using conventional clinical and immunological parameters. The primary outcome measure for biological efficacy will be parotid gland salivary output.

Patients between 18 years of age or older who received radiation treatment for head and neck cancer at least 5 years before enrolling in this study, who have no evidence of recurrent tumor, who have dry mouth and who secrete abnormally low levels of saliva from the parotid glands will be eligible for this study. Candidates will be screened with a medical history, physical examination, blood, urine and saliva tests, electrocardiogram (EKG), chest x-ray, MRI exam, gallium scan (a nuclear medicine test to look for inflammation in the salivary glands), technetium pertechnetate scan (a nuclear medicine test to examine salivary gland function), parotid sialogram (x-ray of parotid gland), PET and CT scans to look for signs of tumor and a skin biopsy to collect skin cells for use in immunological tests.

Participants will be randomly grouped to receive a particular dose of the hAQP1 vector as provided in Table 1 and will undergo ultrasound mediated gene transfer as described above to transfer the hAQP1 vector. Saliva will be collected from the parotid glands at 6 and 24 hours after gene transfer of vector, and ten to 14 days after administration, patients will be admitted to a clinical center for up to 4 days for the following tests and procedures:

On the first day, administration, through a catheter, of the study drug hAQP1 into one parotid gland.

Monitoring over the next 3 days for changes in patients' ability to produce saliva including medical examinations and several blood, urine and saliva collections.

Technetium scan on day 2.

Gallium scan on day 2.

Patients return for follow-up visits at 1, 2, 4, and 6 weeks after the hAQP1 transfer and then 3, 4, 5, 6 and 12 months for a medical examination and blood, urine and saliva collections.

TABLE 1
Dose levels

| | |
|---|---|
| 1 | 2.4 × 10⁷ |
| 2 | 4.8 × 10⁷ |
| 3 | 2.9 × 10⁸ |
| 4 | 1.3 × 10⁹ |
| 5 | 5.8 × 10⁹ |
| 6 | 3.5 × 10¹⁰ |

Patients will be expected to show improvement in saliva production as a result of administration of the hAQP1 gene transfer at all dose levels as studies using adenovirus delivery vectors have shown effective increase in saliva production at all dose levels. Moreover, the use of a non-viral vector is expected to allow for long term improvement in saliva production and allow for re-dosing.

What is claimed is:

1. A method for delivering genetic material to a salivary gland comprising:
contacting a salivary gland with a solution of about 5% to about 30% microbubbles and about 10% to about 25% genetic material; and
applying ultrasound to the salivary gland.

2. The method of claim 1, wherein salivary gland is selected from the parotid salivary gland, the submandibular salivary gland, the sublingual salivary gland or combinations thereof.

3. The method of claim 1, wherein the genetic material is a viral vector.

4. The method of claim 1, wherein the genetic material is a non-viral vector.

5. The method of claim 1, wherein the genetic material comprises a gene from which a therapeutically effective amount of an exogenously expressed protein can be produced.

6. The method of claim 1, wherein preparing a solution comprises combining the genetic material with microbubbles in a physiologically acceptable solution.

7. The method of claim 6, wherein the physiologically acceptable solution comprises a liquid selected from saline, phosphate buffered saline, aqueous glucose, human serum albumin, and combinations thereof.

8. The method of claim 6, wherein the physiologically acceptable solution is phosphate buffered saline.

9. The method of claim 1, wherein the microbubbles comprise a shell selected of a material selected from albumin, lipids, phospholipids, polymers, and combinations thereof.

10. The method of claim 1, wherein the microbubbles comprise a gas selected from air, oxygen, nitrogen, noble gases, sulfur hexafluoride, perfluoride, and combinations thereof.

11. The method of claim 1, wherein the microbubbles have a diameter of from about 1 μm to about 4 μm.

12. The method of claim 1, wherein the solution comprises about 10% to about 20% microbubbles.

13. The method of claim 1, wherein the solution comprises about 15% microbubbles.

14. The method of claim 1, wherein contacting comprises injecting the solution into the salivary gland.

15. The method of claim 1, wherein applying ultrasound comprises applying diagnostic ultrasound, therapeutic ultrasound, focused ultrasound, high intensity focused ultrasound, or combinations thereof to the salivary gland.

16. The method of claim 1, wherein the ultrasound comprises ultrasound energy having an acoustic power density of from about 0.05 W/cm² to about 10 W/cm².

17. The method of claim 1, wherein the ultrasound comprises ultrasound energy having a frequency of from about 0.015 MHz to about 10 MHz.

18. The method of claim 1, wherein applying the ultrasound occurs for a time period of from about 30 seconds to about 2 minutes.

19. The method of claim 1, wherein greater than 20% of the genetic material is delivered to the cytosol of cells of the one or more salivary glands.

20. The method of claim 1, wherein the method is repeated.

21. The method of claim 1, further comprising administering a second form of therapy.

22. A method for treating a disease comprising:
contacting a salivary gland with a solution of about 5% to about 30% microbubbles and about 10% to about 25% of the non-viral vector; and
applying ultrasound to the salivary gland.

23. The method of claim 22, wherein salivary gland is selected from the parotid salivary gland, the submandibular salivary gland, the sublingual salivary gland or combinations thereof.

24. The method of claim 22, wherein the steps of contacting the salivary gland with the solution and applying ultrasound to the salivary gland are repeated at regular intervals for a period of time.

25. The method of claim 22, wherein expression levels of a protein expressed from the non-viral vector are maintained over the period of time as a result of the repeating the steps of contacting the salivary gland with the solution and applying ultrasound to the salivary glands.

26. The method of claim 22, wherein the non-viral vector comprises a therapeutic protein.

27. A method for treating a disease comprising:
contacting a salivary gland with a solution of about 5% to about 30% microbubbles and about 10% to about 25% of the non-viral vector;
applying ultrasound to the salivary gland; and
repeating the steps of contacting the salivary gland with the solution and applying ultrasound to the salivary gland at regular intervals over an extended period of time.

28. The method of claim 27, wherein salivary gland is selected from the parotid salivary gland, the submandibular salivary gland, the sublingual salivary gland or combinations thereof.

29. The method of claim 27, wherein expression levels of a protein expressed from the non-viral vector are maintained over the period of time as a result of the repeating the steps of contacting the salivary gland with the solution and applying ultrasound to the salivary gland.

30. The method of claim 27, wherein the extended period of time comprises greater than 1 year.

31. The method of claim 27, wherein the extended period of time is greater than 5 years.

32. The method of claim 27, wherein the extended period of time is the lifetime of the subject.

33. A pharmaceutical composition comprising:
about 5% to about 30% microbubbles; and
about 10% to about 25% genetic material.

34. The pharmaceutical composition of claim 33, further comprising a physiologically acceptable solution.

35. The pharmaceutical composition of claim 34, wherein the physiologically acceptable solution comprises a liquid selected from saline, phosphate buffered saline, aqueous glucose, human serum albumin, and combinations thereof.

36. The pharmaceutical composition of claim 34, wherein the physiologically acceptable solution is phosphate buffered saline.

37. The pharmaceutical composition of claim 33, wherein the solution comprises about 10% to about 20% microbubbles.

38. The pharmaceutical composition of claim 33, wherein the solution comprises about 15% microbubbles.

39. The pharmaceutical composition of claim 33, wherein the microbubbles comprise a shell selected of a material selected from albumin, lipids, phospholipids, polymers, and combinations thereof.

40. The pharmaceutical composition of claim 33, wherein the microbubbles comprise a gas selected from air, oxygen, nitrogen, noble gases, sulfur hexafluoride, perfluoride, and combinations thereof.

41. The pharmaceutical composition of claim 33, wherein the microbubbles have a diameter of from about 1 μm to about 4 μm.

42. The method of claim 1, wherein said genetic material is COX-1/PGIS transgene.

43. The method of claim 22, wherein said genetic material is COX-1/PGIS transgene.

44. The method of claim 22, wherein said disease is pulmonary arterial hypertension.

45. The method of claim 33, wherein said genetic material comprises an nucleotide sequence corresponding to a protein selected from the group consisting of water channels or an anti-inflammatory cytokine.

46. The method of claim 45, wherein the water channel is aquaphorin-1.

50. The method of claim 45, wherein the anti-inflammatory cytokine is cox-1 or cox-2.
